(19) United States
(12) Patent Application Publication
(10) Pub. No.: US 2021/0212576 A1
(43) Pub. Date: Jul. 15, 2021

(54) Monitoring of Physiological Parameters

(71) Applicant: Alunos AG, Unterageri (CH)

(72) Inventors: William Jack MacNeish, Newport Beach, CA (US); Reto Carrara, Finstersee (CH)

(21) Appl. No.: 16/967,539

(22) PCT Filed: Feb. 22, 2019
(86) PCT No.: PCT/EP2019/054477

§ 371 Date: Aug. 5, 2020

Related U.S. Application Data
(60) Provisional application No. 62/634,684, filed on Feb. 23, 2018.

(51) Int. Cl. A61B 5/0205; A61B 5/00; A61B 5/08; G16H 50/20

(52) U.S. Cl. CPC A61B 5/02055; A61B 5/0059; G16H 50/20; A61B 5/0013; A61B 5/0816; A61B 5/7264

(57) ABSTRACT

Physiological monitoring apparatuses and methods are disclosed. A physiological monitor includes imaging sensors, one of which is a time-of-flight imaging sensor. The physiological monitor also includes a processing device to receive data streams from the imaging sensors. The processing device may then extract time parameter data from the data streams, identify a physiological parameter from the extracted parameter data, and provide an indication of the physiological parameter.

### TECHNICAL FIELD

The present invention relates to the monitoring of physiological parameters of a subject. The invention particularly concerns an apparatus for monitoring physiological parameters of a subject, a method for monitoring physiological parameters of a subject as well as a computer program product for performing the steps of this method.

### PRIOR ART

Monitoring of physiological parameters of a subject may provide insight into that person's health, performance or other status. Physiological monitoring is for example carried out in hospitals and doctor's offices, in order to monitor the development of the health conditions of a patient. Physiological monitoring, however, is not only carried out in patients with poor or at least impaired health, but is also widely used with respect to healthy human and animal subjects. For example, the training progress of athletes or the well-being of healthy and in particular elderly people can be monitored. The monitoring of e.g. the circulatory system is becoming more and more popular nowadays even in healthy humans, because associated health incidents can occur suddenly and with severe consequences.

The monitoring of physiological parameters of a human or animal subject, such as of the circulatory system (e.g. heart rate) or other vitals (temperature or respiration), often requires direct contact of one or several sensors with the monitored subject when using state-of-the-art devices. Depending on the type of monitoring and on the used apparatus, measurements sometimes even have to be made invasively in the prior art, i.e. by inserting a sensor at least partially into or onto the body of the subject. Carrying out measurements which are invasive or which require physical contact is not only unpleasant for the subject, but can also affect the behavior of the subject during the measurement and/or even directly influence the measured parameters, for example when monitoring a patient in a sleep lab. Many physiological monitoring techniques may be invasive to the subject, unfit for certain environments, or lack a reading quality or accuracy for some purposes.

A very popular and widely applied physiological monitoring of healthy humans concerns the surveillance of babies and infants during their sleep. The function of most baby monitors, however, is very simple and often only based on the capturing of sounds and/or images of the baby. The parent is able to remotely hear or view the baby and/or is alerted by the monitoring apparatus, if the baby wakes up and starts to cry. Most of the currently available baby monitors are limited to this simple functionality.

A baby monitor that provides further information concerning the health status of the baby is disclosed in WO 2018/034799 A1. In this document, an apparatus is disclosed which has a time of flight (ToF) sensor, in order to also capture information about e.g. the breathing rate or the heart rate of the baby. A ToF sensor is able to resolve distances with good spatial resolution based on the known speed of light, in order to measure the propagation time of a light signal between the sensor and the subject for each point of e.g. an image. By means of a ToF sensor, the motion of e.g. a patient's or of a baby's body or part of (e.g. torso) can be measured at such high resolution that for example the breathing or heart rate can be determined.

While first applications of ToF sensors for monitoring physiological parameters showed promising results in the prior art, the technique is still not robust and reliably enough to be routinely and widely applied.

### SUMMARY OF THE INVENTION

It is an object of the present invention to provide an apparatus, which not only allows a safe and reliable monitoring of physiological parameters of a subject, but which can also be applied easily.

This object is solved by an apparatus for monitoring physiological parameters of a subject as claimed in claim 1. Further embodiments of the apparatus are provided in dependent claims 2 to 13. A method used for monitoring physiological parameters of a subject is claimed in claim 14 and a computer program product comprising software code portions for performing such a method is provided in claim 15.

The present invention thus provides an apparatus, i.e. a system, for monitoring physiological parameters of a subject, comprising:
a plurality of imaging sensors, wherein at least one of the imaging sensors is a time-of-flight (ToF) imaging sensor; and
a processing device coupled to the plurality of imaging sensors, the processing device being adapted to:
receive data streams from the plurality of imaging sensors;
extract time parameter data from the data streams;
identify a physiological parameter from the extracted time parameter data; and
provide an indication of the physiological parameter from the extracted time parameter data.

The plurality of imaging sensors further comprises a thermal imaging device.

Thus, the apparatus comprises at least two imaging sensors, with one of them being a ToF imaging sensor and the other being a thermal imaging device or sensor. By having a thermal imaging device in addition to the ToF imaging sensor, an easy to handle apparatus for monitoring physiological parameters of a subject is achieved which allows a much safer and more reliable monitoring. The thermal imaging device can for example be used to identify a region-of-interest (ROI) for the analysis of the data of the ToF imaging sensor, which analysis can e.g. be directed to breathing rhythm, cardiac pulsation etc. A possible ROI could for example be an uncovered area of skin which can then be analyzed by the data of the ToF imaging sensor with regard to breathing rhythm, heart rate etc. Alternatively or in addition, the data generated by the thermal imaging device can be used to measure the temperature, e.g. the core temperature, of the subject, in order to for example detect fever and/or hypothermia, in which case e.g. an alert can be generated by the apparatus. It is also conceivable to measure a difference in the temperature between the chest and the extremities of the subject, in order to e.g. obtain information about the subject's blood perfusion. The thermal imaging device can also be used to measure skeletal movements, i.e. gross motion, of the subject. In this way, the thermal imaging device in combination with the ToF imaging sensor can help to detect an ill- or shock-status of the subject by for example combining the temperature data of the thermal imaging device with the data concerning the subject's breathing rhythm as determined by the ToF imaging sensor. In comparison to an apparatus having a ToF imaging sensor only, the provision of a thermal imaging device thus allows an improved analysis of the data generated by the ToF imaging sensor and furthermore allows the generation of additional data concerning the subject's status that can be combined with the data generated by the ToF imaging sensor. By combining the data of the ToF imaging sensor with the data of the thermal imaging device, an indication of the monitored physiological parameter can be obtained that is particularly precise and reliable. The provision of a thermal imaging device in an apparatus with a ToF imaging sensor thus enables a particularly reliable and robust physiological monitoring.

The apparatus is advantageously a non-invasive apparatus, i.e. an apparatus which allows non-invasive monitoring of physiological parameters of a subject. Particularly advantageously, the apparatus is even a contactless apparatus which allows monitoring of physiological parameters of a subject without requiring any physical contact with the subject at any time.

The subject can be a human or animal subject. It can e.g. be a patient, a healthy or sick adult at home or a baby. The apparatus can be a baby monitor or a fertility monitor or be used for the monitoring of patients in a sick room or of elderly people in a nursing, rest or special-care home. It can for example be used to monitor patients suffering under Parkinson's or Alzheimer's disease or suffering under epilepsy. Furthermore, the apparatus can be used for monitoring subjects in a sleep lab. The purpose of the apparatus can particularly be to prevent sudden infant death, sleep apnea and/or cardiovascular disorders.

The indication provided by the processing device based on the extracted time parameter data can for example be the actual heart rate of a patient, an indication about the sleeping-status of a baby, the core temperature of the subject etc. In another embodiment, the indication can be whether the baby is sleeping on its back and/or with the pacifier in its mouth, in order to for example reduce the risk for sudden infant death. In this respect, it is also possible to track eye movements with both closed and open eyes of the baby or of the patient, in order to detect whether the monitored subject is about to sleep in or to wake up. Tracking of eye movements can for example be done by a respective analysis of the data of the ToF imaging sensor within a ROI identified by means of the data of the thermal imaging device and, optionally, in combination with the data of a RGB imaging device (see further below). Furthermore, objects can be taken into account which are in the area of the subject (in particular in the area of the baby) and might be disturbing or even pose a risk to the subject (a pillow, a pet, toys etc.).

The provided indication can lead to an alert being generated by the apparatus, if for example the identified temperature, e.g. core temperature, of the subject drops or exceeds below or above a certain threshold or if a shock-status of the subject is detected that requires immediate treatment or if the subject is about to wake up and/or has already done so.

In other embodiments, the apparatus can also be used, e.g. in a closed loop control, to control a respiration assistance system, such as a medical ventilator. In this case, the data of the ToF or RGB imaging sensor are preferably used alone or in combination with thermal imaging device to detect the breathing rate and/or the breathing volume of the subject.

The thermal imaging device can particularly be adapted to measure infrared-radiation in the near-infrared range, i.e. in a wavelength-range of 780 nm to 3 μm.

The apparatus preferably comprises an illumination component to provide one or more of narrow frequency illumination or structured illumination. Narrow frequency illumination and/or structured illumination allow obtaining even more information about the subject by means of the ToF imaging sensor.

The plurality of imaging sensors preferably further comprises an RGB imaging device. The RBG imaging device can be a two-dimensional (2D) or three-dimensional (3D) camera and is preferably in the form of a CMOS- or CCD-camera. By means of the RGB imaging device further data can be obtained from the subject, such as for example of the skin color of the subject. The data of the RGB imaging device can for example also be used for ROI-identification (as an alternative or in combination with the ROI-identification by means of the data of the thermal imaging device) with respect to the analysis of the data of the ToF imaging sensor. The data of the RGB imaging device can for example also be used to identify skeletal movements and/or to identify, whether the eyes of the subject are open or closed, which is possible for example in combination with ROI-identification by means of the data of the thermal imaging device and/or in combination with the data of the ToF imaging sensor. The further data can thus be combined with the data of the ToF imaging sensor and of the thermal imaging device, in order to further improve reliability and robustness of the monitoring. The RGB imaging device can particularly be adapted for measurements of wave lengths in the range of less than 700 nm. The RGB imaging device can also be adapted for measurements in a restricted range of wave lengths, such as e.g. in the range of visible light (400 to 700 nm), of ultraviolet light (10 to 400 nm) or of infrared light (700 to 1050 nm). The RBG imaging device can also be used to send a video-signal to a remote device, in order to allow e.g. the parents or the medical staff to visually observe the monitored environment.

Particularly robust and reliable results are obtained when combining the data of the ToF imaging sensor, the data of the thermal imaging device and the data of the RGB imaging device for providing an indication of the physiological parameter. In doing so, data related to distance, temperature and color are preferably combined, in order to provide an indication of the physiological parameter. It has turned out that this particular combination of data offers a large variety of possibilities to monitor vital data of a subject in a particularly reliable and robust way.

The apparatus preferably comprises a microphone to receive audio data in an environment monitored by the plurality of imaging sensors. The microphone can be a unidirectional or a combination of multiple microphones, in order to determine directionality. The audio data can be combined with the data of the ToF or RGB imaging sensor and/or with the data of the thermal imaging device, in order to further improve reliability and robustness of the monitoring. Alternatively or additionally, the audio data can be used to acoustically observe the monitored environment by means of a remote device.

To extract time parameter data from the received data streams, the processing device is preferably further adapted to identify an edge in at least one of the data streams and to monitor motion characteristics of the detected edge(s). Thus, the processing device is preferably adapted to carry out edge detection and/or edge tracking which are terms well known in image processing, machine vision and computer vision.

In the context of the present document, the term “time parameter data” generally refers to parameter data which can potentially vary over time. The time parameter data can concern for example a distance, a temperature, a color-value, a sound level or any combinations thereof.

The processing device is preferably also adapted to determine a signal to noise ratio of data in the received data streams and to weight a first data stream higher than a second data stream based on the determined signal to noise ratios. The processing device is advantageously also adapted to combine extracted time parameter data from the data streams based on relative weights of the first data and second data to identify the physiological parameter. It is noted in this respect, that a data stream is usually generated by each of the imaging-sensors. Thus, by means of weighting the data streams based on their signal to noise ratios, a more robust data analysis is obtained, while still taking into account the received information of as many of the plurality of imaging sensors as possible.

For extracting the time parameter data, the processing device is advantageously further adapted to perform pattern recognition to determine movement of an identified pattern in the received data streams. Pattern recognition is a well-known term in image processing and refers to the automated recognition of patterns and regularities in data, such as in imaging data. The pattern recognition is preferably carried out by means of machine learning or artificial intelligence, in particular by means of deep learning. Thus, the processing device is preferably adapted to carry out machine learning or artificial intelligence, in particular deep learning.

The processing device is advantageously adapted to filter a first data stream of one imaging sensor based on a frequency of respiration identified in a second data stream of a second imaging sensor. Alternatively or in addition, the processing device is advantageously adapted to filter a first data stream of one imaging sensor based on a frequency of cardiac pulsation identified in a second data stream of a second imaging sensor.

The physiological parameter is preferably one or more of respiration rate, temperature or heart rate. Respiration rate, temperature, and heart rate are physiological parameters that not only fundamentally characterize the health state of the subject, but also provide indications for example whether the subject is sleeping or not or is about to sleep in or to wake up.

It is, however, also possible for the apparatus to comprise a pulse oximeter device, in order to provide further data about the heart rate of the subject. The data of the pulse oximeter device can be used as an alternative or in addition to the data of the imaging sensors, in order to determine the heart rate of the subject. The apparatus can also comprise an electroencephalogram (EEG)-device, in order to obtain data concerning the brain waves, and/or a flowmeter for measuring the breathing of the subject. The processing device is then preferably adapted to combine the data of the pulse oximeter device and/or of the EEG-device and/or of the flowmeter with the data of the imaging sensors to provide an indication of the physiological parameter.

In a particularly preferred embodiment, the apparatus for monitoring physiological parameters of a subject is adapted to monitor the physiological parameters without contacting the subject.

The plurality of imaging sensors and the processing device can be integrated in a single, preferably compact housing. A display, loudspeaker and/or signal generator can be integrated in the housing, in order to visually and/or acoustically reflect the indication of the physiological parameter provided by the processing device. Alternatively or additionally, the display, loudspeaker and/or signal generator can also be provided on a remote host interface to which the indication of the physiological parameter is transmitted by the processing device, in order to be indicated at a distance from the processing device. The transmission from the processing device to the remote host interface can be a wired or a wireless (for example Wi-Fi or Bluetooth) transmission. For this purpose, the apparatus can comprise a wireless transmission device which can be part of the processing device or which can be coupled to the processing device. The remote host interface can be a computer or a smart phone for example.

The apparatus is preferably adapted to send the data streams, the time parameter data, the physiological parameter and/or the indication of the physiological parameter to a cloud computing infrastructure or directly to the host device. In the cloud computing infrastructure, the received data from a plurality of such apparatuses can be stored, collected and/or processed. The processing of the received data in the cloud computing infrastructure is preferably carried out by means of artificial intelligence, comprising in particular a deep learning or other algorithm. By means of the cloud computing infrastructure, the algorithms of the apparatuses for extracting time parameter data from the data streams, for identifying a physiological parameter and/or for providing an indication of the physiological parameter can be improved using the collected “big data”. The cloud computing infrastructure can particularly be adapted to improve signal processing, in particular image processing, such as edge detection and pattern recognition.

The invention is also directed to a method for monitoring physiological parameters of a subject, in particular by using the apparatus as indicated above. The method comprises at least the method steps as follows:
receiving, by a processing device, a plurality of data streams from each of a plurality of imaging sensors, wherein at least one of the imaging sensors is a time-of-flight imaging sensor;
extracting time parameter data from the data streams;
identifying, by the processing device, a physiological parameter from the extracted time parameter data; and
providing an indication of the physiological parameter from the extracted time parameter data.

The plurality of imaging sensors further comprises a thermal imaging device.

Furthermore, the invention is directed to a computer program product directly loadable into the internal memory of a digital computer, comprising software code portions (e.g. HDL, procedural language, software, firmware, etc.) for performing the method steps of the method as indicated above, when said product is run on a computer. Thus, the software code portions of the computer program product are adapted, when being run on a computer, to carry out the above mentioned method for monitoring physiological parameters of a subject, in particular by using the apparatus as indicated above. Hence, the computer program product is preferably adapted to be loaded into the memory of a computer or of a controller that is used for controlling the apparatus or monitoring physiological parameters of a subject as described above. The computer program product is preferably stored on a storage device readable by a computer. The computer program product thus comprises executable instructions to carry out the method as indicated. Preferably, a non-transitory computer-readable medium is provided comprising the computer program for carrying out the method as indicated.

Thus, the computer program is adapted to carry out central parts of the method as described above when executed in a processor of a computer. Preferably, a computer program product is provided that can be loaded directly into the internal memory of a digital or analog computer and comprises software segments which cause the above-mentioned method to be carried out, when the product is running on a computer. The computer program can be realized as a computer program code element which comprises computer-implemented instructions to cause a processor to carry out a respective method. It can be provided in any suitable form, including source code or object code. In particular, it can be stored on a computer-readable medium or embodied in a data stream. The data stream may be accessible through a network such as the Internet.

Physiological monitoring of various parameters of a subject can be used in a variety of settings to improve the health or well-being of a subject. Monitored physiological parameters may include heart rate, respiration, temperature, blood pressure or other indications of a subject's health or status. Physiological monitoring may be performed in hospitals, doctor's offices, children's cribs, athletic training facilities, homes, elderly care facilities or any other environment where knowledge of a subject's current health parameters could provide additional benefits.

Disclosed herein are apparatuses, i.e. devices, and methods for monitoring physiological parameters of an individual. Although generally described as monitoring of a human subject, apparatuses and methods as described herein could be used to monitor multiple subjects or non-human subjects. Additionally, various configurations as described herein may include different processes or devices that are within the scope described.

In some embodiments, a sensor array may include several sensors that receive signals representing a target subject or monitored environment. For example, a sensor unit may include an optical sensor such as a CMOS camera, a microphone, a thermal imaging device, a time of flight (ToF) imaging device, or the like. In some embodiments, a sensor array may include fewer or additional devices to generate different or additional signals for use in determining and monitoring physiological parameters of target subjects.

In some embodiments, a monitoring apparatus may include one or more illumination devices to be used with one or more of the sensors. For example, a flash or modulated light may be used with a particular frequency of light that a ToF sensor is designed to receive. In some embodiments, structured illumination may also be used with a ToF or RGB (e.g. CMOS or CCD) sensor to provide additional information received through the ToF or RGB sensor.

Data generated by the various sensors in a sensor array may be used to determine one or more physiological parameters of a subject. In some embodiments, the data received from each sensor may be provided continuously or in discrete increments. The sensor array may provide signals from each sensor to a processing device. In some embodiments, the raw data from a sensor array may be pre-processed to reduce noise, shift array values from each sensor to provide alignment, compress data, or otherwise improve the signals received from each sensor.

The processed data may then indicate one or more parameters associated with a monitored subject. For example, the processed data may indicate a region of interest based on color or another parameter from one or more of the sensors. In some embodiments, a monochrome or RGB (e.g. CMOS or CCD) image sensor may be used to identify one or more regions of interest. For example, a region may be identified based on skin color or portions of the image that indicate it is not likely to be associated with the subject. In some embodiments, viewing a specific spectrum such as near UV light may indicate that certain features present in the monitored environment may be clothing, blankets, or other known elements.

The processing device may then use the output data to determine the desired physiological parameters. For example, a pattern recognition service may identify one or more patterns in a first image received from one of the sensors. The pattern recognition service may then attempt to find the same pattern in other received image data. The pattern recognition service can then output a trace of the pattern over time. The processing device can use the movement of the pattern to determine one or more physiological parameters. For example, the processing device may determine respiration or heart rate based on the movement of the signal over time. In some embodiments, the trace provided by the pattern recognition service may be combined with other signals from different sensors to further increase the accuracy of an output physiological parameter measurement. In some embodiments, additional processes may be used such as edge detection with tracked motion. In some embodiments, pattern recognition and edge detection services may be applied to an RGB array, a monochrome array, a ToF sensor, a thermal imaging device, or other devices.

In some embodiments, a sensor array may include a ToF sensor. The ToF sensor provides an array of measurements indicating the distance of various elements in a monitored environment from the imaging sensor. A processing device may then determine one or more regions to monitor in the ToF data. In some embodiments, the region of interests may be determined based on data from another sensor element. For example, a region of interest may be identified based on the imaging data from an RGB or thermal imaging device. The ToF data may then be aligned with the RGB imaging data and the ToF may monitor movement in a region identified as likely to provide an indication of the physiological parameters such as respiration or heart rate. The ToF imaging data may then be filtered and processed. A signal processing system may determine one or more parameters based on movement detected in changing distances in the sensor array.

### SHORT DESCRIPTION OF THE FIGURES

The present disclosure is illustrated by way of example, and not by way of limitation, and can be more fully understood with reference to the following description of preferred embodiments when considered in connection with the figures. In the figures it is shown:

FIG. 1 shows a block diagram depicting an example of a physiological monitoring system operating to monitor a subject, in accordance with some aspects of the present disclosure;

FIG. 2 shows a block diagram depicting an example of a physiological monitoring system operating to monitor a subject, in accordance with some aspects of the present disclosure;

FIG. 3 shows a block diagram depicting an example of a physiological monitoring system operating to monitor a subject, in accordance with some aspects of the present disclosure;

FIG. 4 shows a block diagram depicting an example of data flow in a physiological monitoring system, in accordance with some aspects of the present disclosure;

FIG. 5 shows a block diagram depicting an example of data flow in a physiological monitoring system, in accordance with some aspects of the present disclosure; and

FIG. 6 illustrates an illustrative computer system operating in accordance with one or more aspects of the present disclosure.

### DESCRIPTION OF PREFERRED EMBODIMENTS

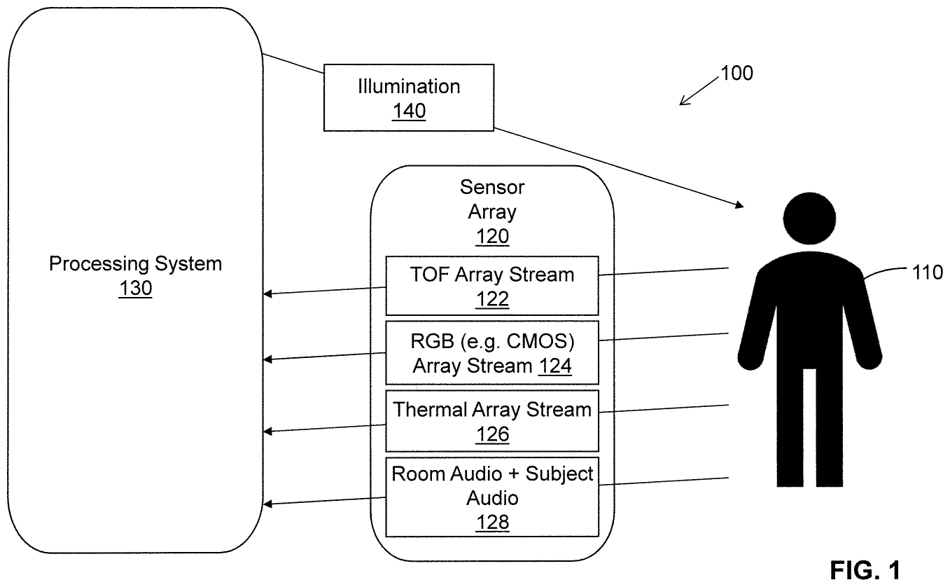

FIG. 1 is a block diagram depicting an example of physiological monitoring system 100 operating to monitor a subject 110. The subject 110 may be an adult in a hospital, a child in a crib or bed, or another subject to monitor. The physiological monitoring system 100 may include a sensor array 120 coupled to a processing system 130. In some embodiments, the sensor array 120 may also include illumination 140. The sensor array 120 may include a ToF array 122, a RGB (e.g. CMOS or CCD) array 124, a thermal array 126, and a microphone 128. In some implementations, the physiological monitoring system 100 may include fewer or additional components than are shown in FIG. 1. For example, some components of the physiological monitoring system 100 may be combined or divided compared to what is shown in FIG. 1. Furthermore, the physiological monitoring system 100 may include additional features such as communication systems, filtering systems, computation engines, additional memory systems, or additional processing systems.

The sensor array 120 provides data streams from each of the sensors in the array to a processing system 130. The processing system 130 may then determine based on a combination of signals from the sensor array one or more physiological parameters. For example, the processing system 130 may determine changes in position, movement, temperature, color, or the like from one or more of the sensors to determine heart rate, respiration, snoring, presence of a subject, or the like. Further description of systems and methods for determining physiological parameters are described below.

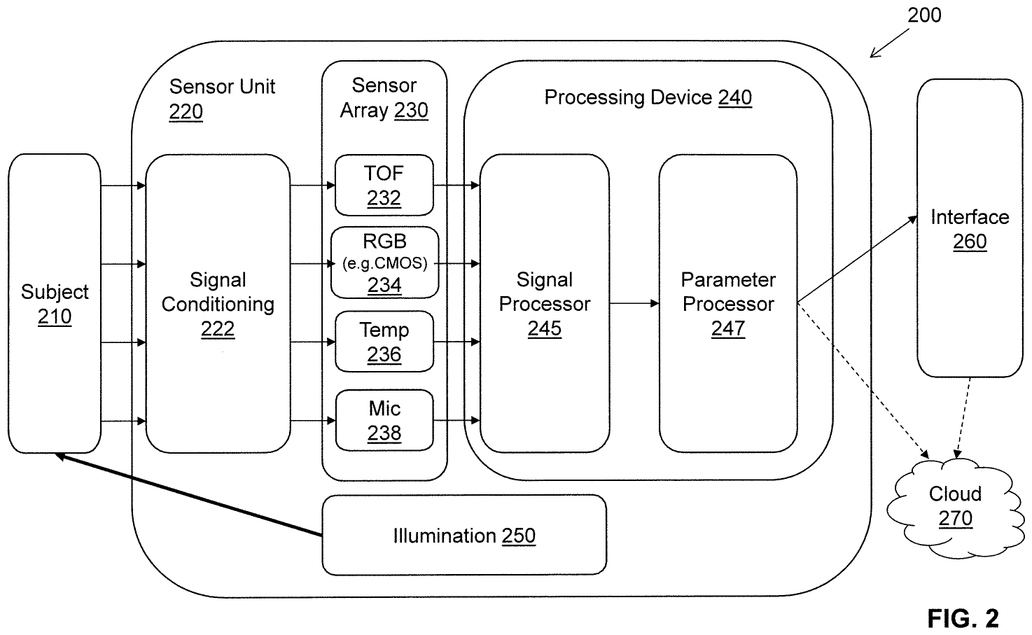

FIG. 2 is a block diagram of an example physiological monitoring system 200. The physiological monitoring system 200 shown in FIG. 2 includes a sensor unit 220 that monitors a subject 210 and provides one or more indications of physiological parameters of the subject 210 to a host interface 260 and/or to a cloud 270, i.e. a cloud computing infrastructure, for monitoring, alerts, storage, record retention, or potential later processing. The sensor unit 220 may include signal conditioning unit 222 that filters or conditions physical signals before they are received by a sensor array 230. The sensor array 230 may include a variety of sensors to sense different physical parameters in the field of view of the sensors and produce electrical signals representing those parameters. Data from the sensor array 230 may be provided to processing device 240. In some embodiments, processing device 240 may include one or more processors. For example, processing device 240 may include one or more single or multicore processors. The data provided by the sensor array 230 may then be processed by the processors 240 to determine one or more physiological properties of the subject 210 to a host interface 260. Preferably, part of the data provided by the sensor array 230 and processed by the processors 240 (e.g. real-time data) is transmitted to the host interface 260 and part of the data provided by the sensor array 230 and processed by the processors 240 (e.g. storage data) is transmitted to the cloud 270. The data and/or time parameters and/or physiological parameters and/or indication of physiological parameters can be transmitted directly from the parameter processor 247 to the cloud 270 or indirectly via the host interface 260. For example, such properties may include presence of a subject, motion of a subject, respiratory data of the subject, cardiac data of the subject, thermal data, position of the subject, or other data that may be useful to a user of a host interface monitoring a subject 210 using the physiological monitoring system 200.

Beginning with the sensor unit 220, the signal conditioning unit 222 may include one or more physical elements that condition information being received by the sensor unit 220 before it is sensed by the sensor array 230. For example, such conditioning may include one or more lenses that condition light waves to be received by the sensor array 230. Such lensing may focus light to direct it at one or more of the sensors in the sensor array 230, may filter out certain frequencies of light to improve signals generated by one or more of the sensors in the sensor array 230, or otherwise condition light to improve performance of the sensor array 230. The signal conditioning unit 222 may also perform other functions such as audio condensing, acoustic filtering, thermal filtering, or otherwise conditioning physical signals received at sensor unit 220.

Sensor array 230 may include multiple sensors that produce signals based on physical signals such as electromagnetic waves, acoustic waves, or the like received at the sensor unit 220 from the subject 210. In the example embodiment shown in FIG. 2, the sensor array includes a ToF imaging device 232, a RGB (e.g. CMOS or CCD) imaging device 234, a thermal imaging device (e.g., an infrared sensor) 236, and a microphone 238. In various embodiments, sensor array 230 may include fewer or additional sensors than shown in FIG. 2. For example, there may be a RGB imaging device and a monochromatic imaging device. In some embodiments, the sensor array 230 may not include a microphone 238 or one of the other sensing devices present in the sensor array 230 as shown in FIG. 2. Each of the sensors in the sensor array 230 may generate an electronic signal to provide from the sensor array 230 to processing device 240. The signals may be provided from the sensor array 230 in a parallel or series connection to the processing device 240. In some embodiments, one or more of the sensing devices in the sensor array 230 may filter or otherwise condition a signal or parameter prior to providing a signal to the processing device 240.

In some embodiments, the sensor unit 220 may also include illumination 250. Illumination 250 may provide constant or pulsed light at particular frequencies to improve detection of certain image qualities by sensor array 230. For example, a pulse of light may be provided at a frequency to be detected by a ToF imaging device in order to provide timing for the ToF imaging device to determine position and distance from the imaging device to one or more features of the subject 210 or the surrounding environment. In some embodiments, a new UV illumination may be provided to improve reflection of UV light to be detected by an image sensor. Structured light may also be provided by illumination 250 in order to provide additional information after detection by a RGB sensor or other imaging device.

The processing device 240 may include one or more processors that determine physiological parameters based on the signals received from sensor array 230. As shown in FIG. 2, the processing device 240 includes a signal processor 245 which can have the form of an image processor and a parameter processor 247. In some embodiments, there may be additional processors including in processing device 240. For example, the processing device 240 may include a communication processor for communicating with a host interface 260.

As shown in FIG. 2, the signal processor 245 may receive signals from sensors in the sensor array 230. The signal processor 245 may then determine a variety of features present in one or more of the sensing device signals. For example, the signal processor 245 may determine a location of chest movement of the subject 210, other regions of movement, temperature changes in different portions of a thermal image, audio signal location and intensity received by a microphone, movement or positions of recognized patterns in a signal, movement of detected edge locations or detected blobs (in color images, spatial images, thermal images, or the like). In some embodiments, there may be multiple signal processors 245. For example, there may be signal processors 245 to determine features from each of the sensors in sensor array 230. Data from the signal processor 245 may be provided to a parameter processor 247.

A parameter processor 247 may interpret the output of the signal processor 245 to determine one or more physiological parameters of a subject 210. For example, the signal processor 245 may provide to the parameter processor 247 indications of movement at one or more different locations in imaging data received from the sensor array 230. The parameter processor 247 may then interpret those indications to determine one or more physiological parameters. For example, if a first data stream from a first sensor in sensor array 230 is processed by the signal processor 245 to indicate movement detected in the imaging data received from the first sensor, the parameter processor 247 may determine a frequency of that movement. For example, breathing may occur at a predictable rate over time. The parameter processor 247 may determine a magnitude of movement identified by the image processor, filter the movement by a known range of frequencies that are in the range of the subject's respiration, and determine an estimated rate of respiration from the movement. In some embodiments, the estimated respiration rate may be compared to those detected in other data streams from other sensors in sensor array 230 to improve the estimated rate of respiration. Similar techniques can be performed to identify volume during respiration, pulse rate, skeletal movement of the individual, or other physiological parameters.

The data provided by the processing device 240 may be shown on a display device coupled to the sensor unit 220, or may be transmitted to another device, such as host interface 260. Host interface 260 may be a smartphone, smart watch, browser on a computer, dedicated interface, tablet, or other device that can provide physiological data directly to a user. In some embodiments, host interface 260 may also include an alert system that provides an alert in response to one or more physiological parameters falling within a specified range. For example, respiration or heart rate above or below a threshold, presence of an unexpected subject 210, thermal changes to a monitored subject 210, or other changes that indicate a potential improvement or decline in the status of a monitored subject 210. In some embodiments, a host interface 260 may provide selected physiological parameters for monitoring based user selection or changes to status of a subject 210. In some embodiments, the host interface 260 may provide data to networked storage location (e.g. cloud 270) for comparison of changes to physiological parameters associated with the subject 210 or compared to other subject monitored by the same or a different physiological monitoring system 200.

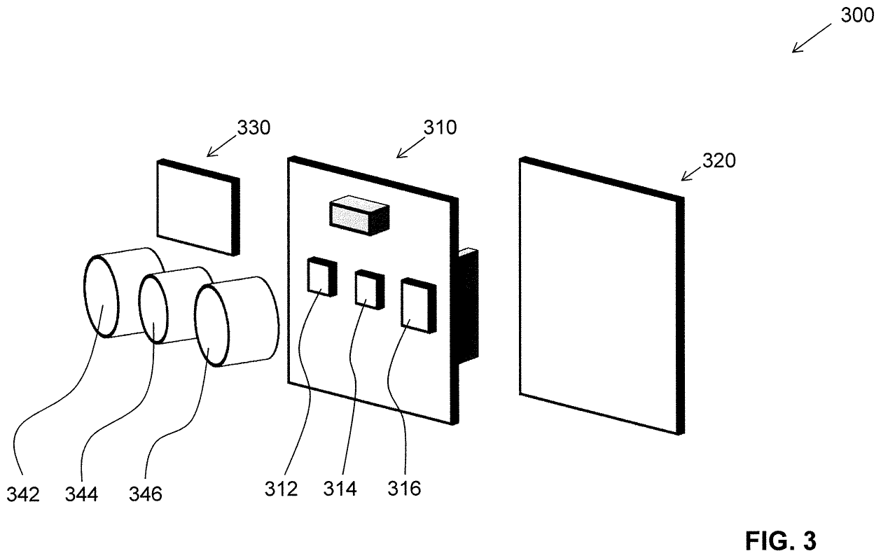

FIG. 3 is a block diagram showing features of a physiological monitor 300 according to some aspects of the present disclosure. The physiological monitor 300 includes a sensor array 310, a processing component 320, lensing mechanisms 342, 344, and 346 and illumination components 330. The example embodiment as shown in FIG. 3 includes a particular physical configuration of a physiological monitor 300. In some embodiments there may be fewer or additional components. Furthermore, the components may be configured differently than shown. For example, in some embodiments, the processing component 320 may include one or more processing devices. Furthermore the processing component 320 may be integrated into a single integrated circuit or a single printed circuit with sensor array 310.

In some embodiments, the physiological monitor 300 may be the same or similar as that described with reference to FIGS. 1 and 3 above. For example, components of physiological monitor 300 may perform the same or similar functions as those described with reference to physiological monitor 200 in FIG. 2.

The sensor array 310 may include a ToF imaging device 312, a RGB (e.g. CMOS or CCD) imaging device 314, and a thermal imaging device 316. The imaging devices may be configured on a PCB that couples the imaging device to power sources (not shown), control systems (not shown), processing component 320, or the like. In some embodiments, the sensor array may include fewer or additional sensors. For example, the sensor array 310 may include a microphone or multiple microphones, additional sensing devices, or other image devices. As shown in FIG. 3, each of the imaging devices has one or more optical systems, such as in particular lenses, to condition light received at the sensor array 310. As shown in FIG. 3, there is a first optical system in the form of a ToF lens 342 (for the ToF sensor 312), a second optical system in the form of a CMOS lens 344 (for the RGB imaging device 314), and a third optical system in the form of a thermal lens 346 (for the thermal imaging device 316). One or more of the optical systems may act as filters to provide certain frequencies of light to the particular imaging devices, a lens to focus light to the imaging devices, or other functions to provide appropriate quantities and spectrums of light to each imaging device. In some embodiments, one or more of the imaging devices on sensor array 310 may not have an optical system to provide conditioning.

The physiological monitor 300 may also include one or more illumination components 330. The illumination components 330 may provide light in pulses at specific frequencies, constant light at a pre-determined wavelength to be used by an imaging device, structured illumination to increase the data present in signals generated by the imaging devices, or the like.

The imaging devices present on the sensor array 310 may be aligned mechanically or through optical image processing. For example, the imaging devices may be coupled to flex components of a PCB and aligned during a calibration stage of processing. The imaging device may be aligned by using a target at a set distance that will show up in spectrums that cause signals in each of the imaging devices, for instance. Thus, the PCB may provide mechanical alignment by aligning each of the imaging devices on a shared target. In some embodiments, the imaging devices may be aligned using one or more image processing techniques. For example, while in use monitoring a subject, the processing component 320 may identify motion or objects identified in data streams from each of the imaging devices to align and focus the imaging devices. Aligning the imaging devices (mechanically or computationally) can enable the processing component 320 to combine data streams from each of the imaging devices to improve reliability, accuracy and types of physiological monitoring that are available.

The processing component 320 may include one or more processing devices as described with reference to processing device 240 of FIG. 2. The processing component 320 may receive signals from imaging devices on sensor array 310 and determine one or more physiological parameters based on analysis of the provided data streams. In some embodiments, the processing component 320 may be on a separate PCB from the sensor array 310, or may be provided as part of the sensor array 310. The processing components may comprise microprocessors, field programmable gate arrays (FPGAs), application specific integrated circuits (ASICs), or other processing components designed or appropriate to determine physiological parameters from signals received from sensing devices. In some embodiments, the processing component 320 may also include one or more filters, digital converters, amplifiers, or other signal conditioning components to improve signal quality before processing by signal, e.g. image, processing devices. The processing component 320 may also include communication systems for providing monitored physiological parameters, or other data, to one or more host interfaces or other recipients.

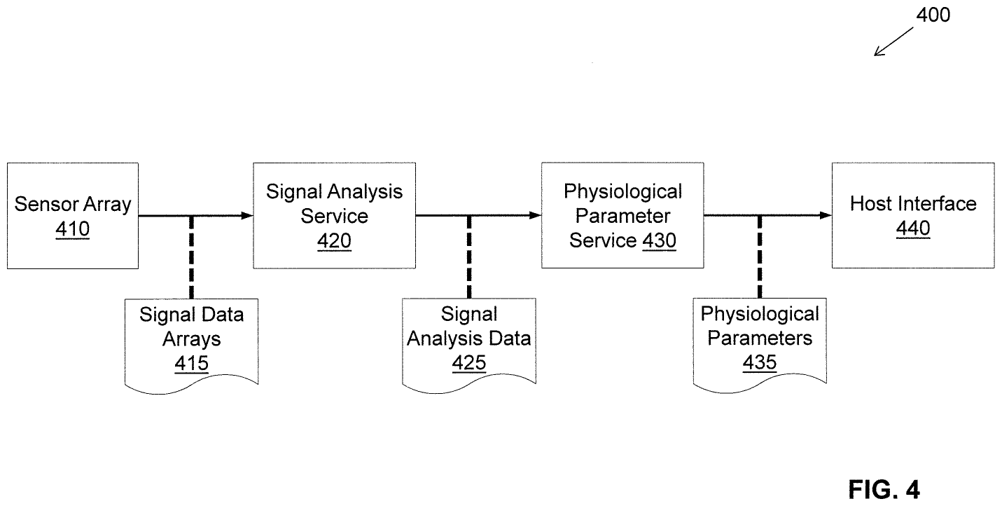

FIG. 4 is a block diagram showing a data flow within a physiological monitor 400 from a sensor array 410 to a host interface 440. The example physiological monitor 400 shown in FIG. 4 includes various components that may be combined or further divided in various embodiments. For example, signal analysis service 420 and physiological parameter service 430 may be combined to process data from at least some data streams received from sensor array 410.

The sensor array 410 may be as described with reference to FIGS. 1, 2, and 3 as described above and include various imaging sensors, audio sensors, temperature sensors, or the like. The sensor array 410 may provide multiple data streams representing data sensed at each sensing device. For example, a ToF sensor may provide a distance array showing the distance of different pixels from the imaging device. A RGB sensor may provide a color and/or intensity array for pixels sensed by the RGB sensor. A thermal sensor may provide a temperature array of different temperatures sensed by the thermal sensor. A microphone or multiples microphones may provide mono, stereo or mutlichannel audio data that may provide sounds as well as directional or position properties of sensed audio.

In some embodiments, ToF sensors, RGB sensors, thermal sensors, or other imaging sensors may provide a data stream of quarter video graphics array (QVGA) quality, video graphics array (VGA) quality, or another video quality. The array may also provide a distance array at 10 fps, 20 fps, 30 fps, 60 fps, or at another frame rate depending on the ToF sensor or frame rates for particular physiological parameters. Audio sensors may provide data at 48 kHz and at different qualities, such as 8 bit or higher. These data streams, or others, may be provided from the sensor array 410 to the signal analysis service 420 in one or more signal data arrays 415. For example, the data streams may be provided as a single stream with each of the data streams from different sensors encapsulated, or may be provided as multiple messages or data streams from each sensor in sensor array 410 to signal analysis service 420.

The signal analysis service 420 may perform image analysis on one or more sensor data streams received. While described as image analysis, it will be understood that audio analysis or other sensor analysis may also be performed. The image analysis service may perform de-noising, frame shifting or scaling, parameter extraction, compression, pattern recognition, movement detection, or the like. In some embodiments, the image analysis system may also combine signals from one or more sensors to identify correlated movement, position, or other data during image analysis.

The signal analysis data 425 output by the signal analysis service 420 may include chest movement (including frequency, magnitude, or the like), overall movement of the subject, temperature detection of portions of the subject, audio signals including location or intensity of audio, skin position movement or color change, edge movement, pattern position, or other image analysis parameters.

The signal analysis data 425 may be provided to a physiological parameter service 430 that determines one or more physiological parameters based on the received signal analysis data 425. For example, the physiological parameter service 430 may extract time-wise parameter data, weight signals received from different data streams, perform averaging of one or more signals, determine image analysis data with low predictive qualities, or the like. For example, if the physiological parameter service 430 includes a number of different data streams with different indications of respiration rates, one or more of those may be ignored if it is more than a threshold different than those predicted by another data stream as it may be monitoring a different motion. Furthermore, predicted physiological parameters may be averaged between different data streams.

In some embodiments, a respiration rate or heart rate detected in one data stream may be used to filter data in a different data stream to improve the accuracy of a measurement. For example, if ToF imaging data is analyzed to predict a respiration rate, that rate may be used to filter motion in RGB data to confirm the rate. Additional physiological parameters may also be generated by analysis of the signal analysis data 425.

The determined physiological parameters 435 may then be provided to a host interface 440. The host interface 440 may be integral to or separate from the imaging sensor 410, the signal analysis service 420, or the physiological parameter service 430. In some embodiments, the determined physiological parameters 435 may be provided as a continuous stream. In some embodiments, the physiological parameters 435 may be provided when a threshold is met, periodically, on request from a host interface 440, or during other time periods. In some embodiments, the physiological parameters may be provided with one or more data streams enabling the host interface to show a representation of one or more of the sensor outputs from sensor array 410.

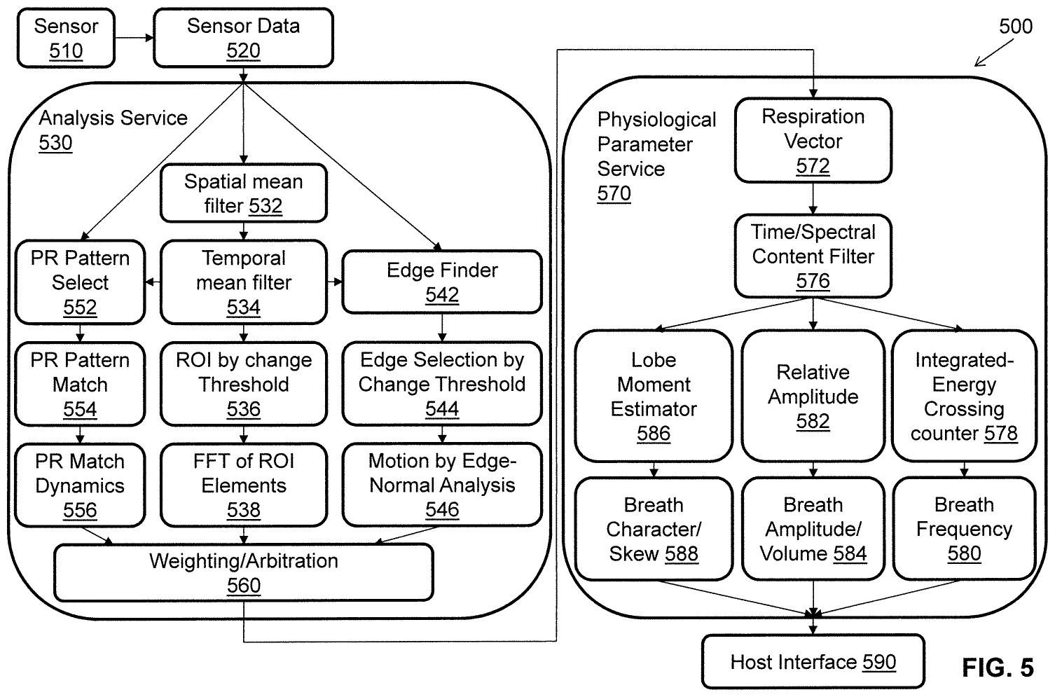

FIG. 5 is a block diagram showing data flow through a physiological monitoring system 500. The physiological monitoring system 500 includes a sensor 510, sensor data 520, an (image) analysis service 530, and a physiological parameter service 570. The physiological monitoring system 500 may provide an output of one or more physiological parameters to a host interface 590. The sensor 510 may be one or more sensors, or a sensor array, as described with reference to FIGS. 1-4 above. The analysis performed by one or more components of the physiological monitoring system 500 may be described with reference to a single data stream from a single image, but may also be performed on multiple data streams received from multiple sensors. The sensor data 520 may be similar or the same as signal data array 415 as described with reference to FIG. 4. For example, the sensor data 520 may include one or more arrays of pixels provided from one or more imaging devices or other sensors.

The sensor data 520 may be provided to the image analysis service 530. In some embodiments, the image analysis service 530 may be the same or similar as the signal analysis service 420 as described with reference to FIG. 4. The image analysis service 530 may include a number of analysis systems as shown that perform different filtering, analysis, and other processes. In some embodiments, the image analysis service 530 may include fewer or additional components than shown in FIG. 5. The data paths and organization of the components may also be different than shown.

In some embodiments, the image analysis service 530 may include a spatial mean filter 532 that provides a blending between pixels in a frame to remove noise from sensor data 520. The output of the spatial mean filter 532 may also be processed by a temporal mean filter 534. The temporal mean filter 534 may average pixels over time to reduce noise from one frame to another. The output of the temporal mean filter 534 and spatial mean filter 532 may be provided to an edge finder 542, a pattern recognition pattern select 552, and a region of interest identifier 536. In some embodiments, one or more of the edge finder 542, the pattern recognition pattern select 552, and the region of interest identifier 536 may receive sensor data 520 without processing by a temporal mean filter 534 or a spatial mean filter 532.

The pattern recognition pattern select 552 may select a pattern that is present in sensor data 520. For example, the pattern select 552 may identify a pattern in imaging data based on well-defined regions, regions with particular reflective characteristics, or other regions or patterns that can be tracked through multiple frames of imaging data. The pattern selected by the pattern select 552 may be provided to a pattern recognition pattern match 554. In some embodiments, patterns may not be selected from each set of sensor data 520 and may instead be provided only periodically to a pattern match 554. The pattern match 554 may walk the pattern through pixels present in sensor data 520 to identify regions that match the selected pattern at least above a threshold amount. Based on comparison of pattern matches provided by pattern match 554 to pattern match dynamics 556, the pattern match dynamics 556 may determine motion such as size changes, translations, or other movements of the pattern with frames in sensor data 520. The changes in in the position or orientation identified by pattern match dynamics 556 may then be used to determine motion in part of sensor data 520.

A region of interest threshold identifier 536 may also receive data from a temporal mean filter 534. Region of interest threshold identifier 536 may identify areas with changes above a threshold value. For example, in ToF sensor data, the region of interest threshold identifier may determine changes in the distance of certain pixels in an array over time. Those that change over a threshold amount may be used by a region of interest threshold identifier 536 to identify regions of interest. In some embodiments, identified regions may be correlated to other sensor data 520 that is received from other sensors 510. For example, a region of interest identified in one set of sensor data may be used to identify an area of interest in a correlated position in another set of sensor data. The region of interest identified by the region of interest identifier 536 may then be provided to a fast Fourier transformation 538 or other transformation to frequency domain to determine one or more frequencies of movement present in the sensor data 520. Any correlated regions of interest in other sensor data arrays may also determine frequencies present in regions of interest.

Edge finder 542 may identify one or more edges present in sensor data 520. For example, an edge may be identified based on changes in contrast of colors in RGB or monochromatic data, changes in distance measured by a ToF imaging device, or other edges indicating a change between elements in detected in sensor data 520. An edge selection threshold 544 may then be applied to determine edges that have a change over a set of pixels in an array over a set threshold. The selected edges may then be analyzed by tracing the edge between frames to determine motion of the edge by an edge motion analyzer 546. The edge motion analyzer 546 may determine motion by performing frequency domain analysis of the edge's position, filtering movement in the edges position, or other tracking of the edges position.

Outputs from the pattern recognition analysis, the region of interest analysis, and the edge detection analysis may be provided to a weighting system 560 to determine weights to provide to each analysis output. In some embodiments, the weighting system 560 may also weigh outputs of analysis provided based on different sensor data streams 520. For example, the weighting system 560 may determine whether certain data streams (ToF, thermal, RGB, monochromatic, audio, or the like) are providing stronger signals for determining physiological parameters. For example, the weighting system may determine signal to noise ratios, consistency between measurements, or other considerations to determine how much weight to provide to different measurements. The weighting system 560 may then provide one or more outputs of the image analysis service 530 to the physiological parameter service 570. For example, the output of the analysis service 530 may be the same or similar as described with reference to the signal analysis data 425 as described with reference to FIG. 4.

The physiological parameter service 570 may determine one or more physiological parameters to output to a host interface 590. For example, the physiological parameters may be similar to those described with respect to FIGS. 1-4 above. While described generally below with respect to respiration, in some embodiments, the physiological parameter service 570 may also provide physiological parameters related to heart rate, blood pressure, or other physiological parameters.

The physiological parameter service 570 may receive an output of the image analysis service 530 at a respiration vector generator 572. The respiration vector generator 572 may combine the output signal of image analysis service 530 according to provided weights. Those weights may be combined into a vector function describing respiration parameters. The output vector may be analyzed by a time and spectral content filter 576 to reduce noise in the combined data.

The filtered respiration vector may then be analyzed to determine breath frequency, breath amplitude or volume, and breath character or skew. The respiration vector, or additional data from image analysis service 530 may be provided to an energy crossing counter 578 that determines a number of times a movement in the respiration vector crosses a set threshold. The counter may then determine a number of breaths recorded within a set period of time to estimate a breath frequency 580. A relative amplitude estimator 582 may also analyze the respiration vector to estimate a volume or amplitude of breath by a subject. For example, in some embodiments, the amplitude estimator 582 may determine an amount of recorded movement based on comparison of the size of a known object in sensor data 520 compared to the measured relative movement in the respiration vector. In some embodiments, the amplitude estimator 582 may compare breath volume over time for a monitored subject. For example, the amplitude estimator 582 may determine whether breath volume is increasing or decreasing over time. The breath amplitude or volume 584 may be provided as a relative value compared to historic measurements for the subject or may be given as an estimate for actual breath volume for the monitored subject. A lobe moment estimator 586 may also receive one or more respiration vectors and determine estimated movement of lobes of a monitored subject. For example, the lobe moment estimator 586 may determine based on one or more movement or motions vectors determined from one or more sensor data 520 direction of lung expansion during respiration. That movement may determine if part of a monitored subjects lungs are working better or worse than others. Thus, a physiological parameter service 570 may provide breath characteristics or breath skew 588.

In some embodiments, fewer or additional physiological parameters may be provided by a physiological monitoring system 500 to a host interface 590. For example, fewer or additional characteristics of a subject's respiration may be provided. In addition, in some embodiments, additional parameters such as blood pressure, pulse, temperature, skeletal movements, or other movements of a subject may be provided based on analysis of one or more sensors 510 in a sensor array of the physiological monitoring system 500.

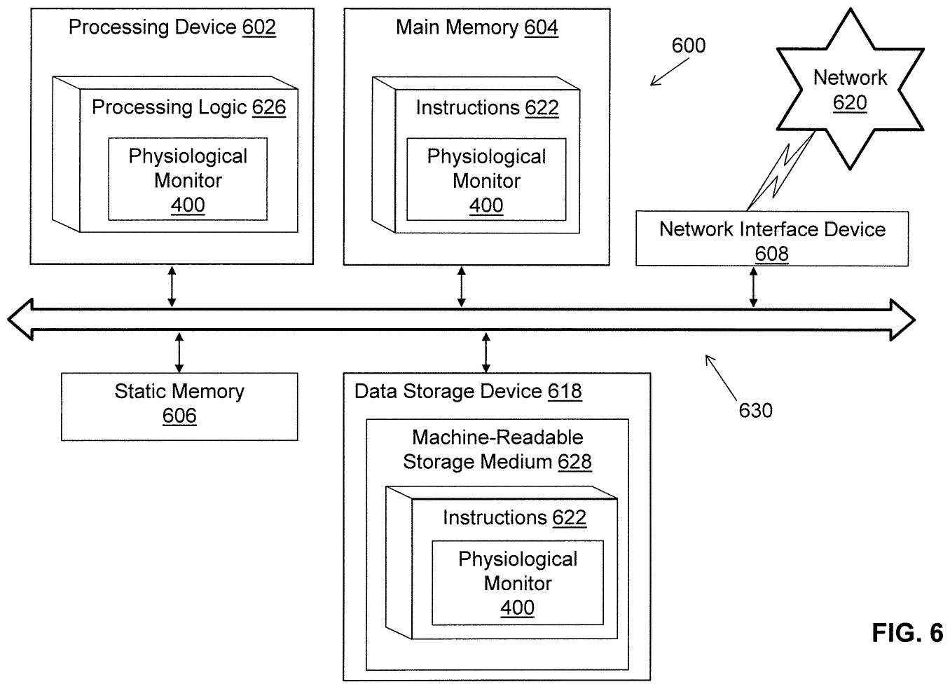

FIG. 6 illustrates a diagrammatic representation of a machine in the example form of a computer system 600 within which a set of instructions, for causing the machine to perform any one or more of the methodologies discussed herein, may be executed. In alternative embodiments, the machine may be connected (e.g., networked) to other machines in a local area network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, a switch or bridge, a hub, an access point, a network access control device, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. In one embodiment, computer system 600 may be representative of a monitoring apparatus, such as a physiological monitoring system as described with reference to FIGS. 1-5 configured to perform account access monitoring or system 400.

The exemplary computer system 600 includes a processing device 602, a main memory 604 (e.g., read-only memory (ROM), flash memory, dynamic random access memory (DRAM), a static memory 606 (e.g., flash memory, static random access memory (SRAM), etc.), and a data storage device 618, which communicate with each other via a bus 630. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses.

Processing device 602 represents one or more general-purpose processing devices such as a microprocessor, central processing unit, or the like. More particularly, the processing device may be complex instruction set computing (CISC) microprocessor, reduced instruction set computer (RISC) microprocessor, very long instruction word (VLIW) microprocessor, or processor implementing other instruction sets, or processors implementing a combination of instruction sets. Processing device 602 may also be one or more special-purpose processing devices such as an application specific integrated circuit (ASIC), a field programmable gate array (FPGA), a digital signal processor (DSP), network processor, or the like. The processing device 602 is configured to execute processing logic 626, which may be one example of system 400 shown in FIG. 4, for performing the operations and steps discussed herein.

The data storage device 618 may include a machine-readable storage medium 628, on which is stored one or more set of instructions 622 (e.g., software) embodying any one or more of the methodologies of functions described herein, including instructions to cause the processing device 602 to execute physiological monitoring system 600. The instructions 622 may also reside, completely or at least partially, within the main memory 604 or within the processing device 602 during execution thereof by the computer system 600; the main memory 604 and the processing device 602 also constituting machine-readable storage media. The instructions 622 may further be transmitted or received over a network 620 via the network interface device 608. The storage device 618 can also be used to store calibration data which occur during the calibration of the plurality of imaging sensors when the apparatus is e.g. initialized or re-started.

The machine-readable storage medium 628 may also be used to store instructions to perform a method for physiological monitoring, as described herein. While the machine-readable storage medium 628 is shown in an exemplary embodiment to be a single medium, the term “machine-readable storage medium” should be taken to include a single medium or multiple media (e.g., a centralized or distributed database, or associated caches and servers) that store the one or more sets of instructions. A machine-readable medium includes any mechanism for storing information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; or another type of medium suitable for storing electronic instructions.

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular embodiments may vary from these exemplary details and still be contemplated to be within the scope of the present disclosure.

Additionally, some embodiments may be practiced in distributed computing environments where the machine-readable medium is stored on and or executed by more than one computer system. In addition, the information transferred between computer systems may either be pulled or pushed across the communication medium connecting the computer systems.

Embodiments of the claimed subject matter include, but are not limited to, various operations described herein. These operations may be performed by hardware components, software, firmware, or a combination thereof.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent or alternating manner.

The above description of illustrated implementations of the invention is not intended to be exhaustive or to limit the invention to the precise forms disclosed. While specific implementations of, and examples for, the invention are described herein for illustrative purposes, various equivalent modifications are possible within the scope of the invention, as those skilled in the relevant art will recognize.

It will be appreciated that variants of the above-disclosed and other features and functions, or alternatives thereof, may be combined into may other different systems or applications. Various presently unforeseen or unanticipated alternatives, modifications, variations, or improvements therein may be subsequently made by those skilled in the art which are also intended to be encompassed by the following claims. The claims may encompass embodiments in hardware, software, or a combination thereof.

Furthermore, the following items are particularly indicated:

Item 1.
A system comprising:
   a plurality of imaging sensors, wherein at least one of the imaging sensors is a time-of-flight imaging sensor; and
   a processing device coupled to the plurality of imaging sensors, the processing device to:
   receive data streams from the plurality of imaging sensors;
   extract time parameter data from the data streams;
   identify a physiological parameter from the extracted time parameter data; and
   provide an indication of the physiological parameter from the extracted time parameter data.

Item 2.
The system of item 1, further comprising an illumination component to provide one or more of narrow frequency illumination or structured illumination.

Item 3.
The system of item 1, wherein the plurality of imaging sensors further comprise an RGB imaging device and a thermal imaging device.

Item 4.
The system of item 1, further comprising a microphone to receive audio data in an environment monitored by the plurality of imaging sensors.

Item 5.
The system of item 1, wherein to extract time parameter data from the data streams, the processing device is further to:
   identify an edge in at least one of the data streams; and
monitor motion characteristics of the detected edge.

Item 6.
The system of item 1, wherein the processing device is further to:
   determine signal to noise ratio of data in the received data streams; and
   weight a first data stream higher than a second data stream based on the determined signal to noise ratios; and
   combine extracted time parameter data from the data streams based on relative weights of the first data and second data to identify the physiological parameter.

Item 7.
The system of item 1, wherein to extract time parameter data, the processing device is further to perform pattern recognition to determine movement of an identified pattern in the received data streams.

Item 8.
The system of item 1, wherein the processing device is further to filter a first data stream of one imaging sensor based on a frequency of respiration identified in a second data stream of a second imaging sensor.

Item 9.
The system of item 1, wherein the physiological parameter is one or more of respiration rate or heart rate.

Item 10.
A method comprising:
   receiving, by a processing device, a plurality of data streams from each of a plurality of imaging sensors, wherein at least one of the imaging sensors is a time-of-flight imaging sensor;
   extracting time parameter data from the data streams;
   identifying, by the processing device, a physiological parameter from the extracted time parameter data; and
   providing an indication of the physiological parameter from the extracted time parameter data.

Item 11.
The method of item 10, further comprising illuminating a subject with one or more of narrow frequency illumination or structured illumination.

Item 12.

The method of item 10, wherein the plurality of imaging sensors further comprise an RGB imaging device and a thermal imaging device.

Item 13.
The method of item 10, further comprising receiving audio data from a microphone in an environment monitored by the plurality of imaging sensors.

Item 14.
The method of item 10, wherein extracting time parameter data from the data streams further comprises:
   identifying an edge in at least one of the data streams; and
   monitoring motion characteristics of the detected edge.

Item 15.
The method of item 10, further comprising:
   determining signal to noise ratio of data in the received data streams; and
   weighting a first data stream higher than a second data stream based on the determined signal to noise ratios; and
   combining extracted time parameter data from the data streams based on relative weights of the first data and second data to identify the physiological parameter.

Item 16.
The method of item 10, wherein extracting time parameter data further comprises performing pattern recognition to determine movement of an identified pattern in the received data streams.

Item 17.
The method of item 10, further comprising filtering a first data stream of one imaging sensor based on a frequency of respiration identified in a second data stream of a second imaging sensor.

Item 18.
The method of item 10, wherein the physiological parameter is one or more of respiration rate or heart rate.

Item 19.
A non-transitory computer-readable medium having instructions stored thereon that, when executed by a computer processing device, cause the computer processing device to:
   receive a plurality of data streams from each of a plurality of imaging sensors, wherein at least one of the imaging sensors is a time-of-flight imaging sensor;
   extract time parameter data from the data streams;
   identify, by the processing device, a physiological parameter from the extracted time parameter data; and
   provide an indication of the physiological parameter from the extracted time parameter data.

Item 20.
The non-transitory computer-readable medium of item 19, wherein the instructions further cause the computer processing device to activate an illumination component to illuminate a subject with one or more of narrow frequency illumination or structured illumination.

REFERENCE NUMERALS

| | | | |
|---|---|---|---|
| 100 | Physiological monitoring system | 126 | Thermal array |
| 110 | Subject | 128 | Microphone(s) |
| 120 | Sensor array | 130 | Processing system |
| 122 | ToF array | 140 | Illumination |
| 124 | RGB (e.g. CMOS) array | | |
| 200 | Physiological monitoring system | 236 | Thermal imaging device |
| 210 | Subject | 238 | Microphone |
| 220 | Sensor unit | 240 | Processing device |
| 222 | Signal conditioning unit | 245 | Signal processor |
| 230 | Sensor array | 247 | Parameter processor |
| 232 | ToF imaging device | 250 | Illumination |
| 234 | RGB (e.g. CMOS) imaging | 260 | Host interface |
| | device | 270 | Cloud |
| 300 | Physiological monitor | 316 | Thermal imaging device |
| 310 | Sensor array | 320 | Processing component |
| 312 | ToF imaging device | 342 | ToF lens |
| 314 | RGB (e.g. CMOS) imaging | 344 | CMOS lens |
| | device | 346 | Thermal lens |
| 330 | Illumination components | | |
| 400 | Physiological monitor | 425 | Signal analysis data |
| 410 | Sensor array | 430 | Physiological parameter |
| 415 | Signal data arrays | | service |
| 420 | Signal analysis service | 435 | Physiological parameters |
| | | 440 | Host interface |
| 500 | Physiological monitoring system | 556 | PR match dynamics |
| 510 | Sensor | 560 | Weighting/arbitration |
| 520 | Sensor data | 570 | Physiological parameter |
| 530 | Image analysis service | | service |
| 532 | Spatial mean filter | 572 | Respiration vector |
| 534 | Temporal mean filter | 576 | Time/spectral content filter |
| 536 | ROI by change threshold | 578 | Integrated-energy crossing |
| 538 | FFT of ROI elements | | counter |
| 542 | Edge finder | 580 | Breath frequency |
| 544 | Edge selection by change | 582 | Relative amplitude |
| | threshold | 584 | Breath amplitude/volume |
| 546 | Motion by edge-normal analysis | 586 | Lobe moment estimator |
| 552 | PR pattern select | 588 | Breath character/skew |
| 554 | PR pattern match | 590 | Host interface |
| 600 | Computer system | 620 | Network |
| 602 | Processing device | 622 | Instructions |
| 604 | Main memory | 626 | Processing logic |
| 606 | Static memory | 628 | Machine-readable storage |
| 608 | Network interface device | | medium |
| 618 | Data storage device | 630 | Bus |

1. An apparatus for monitoring physiological parameters of a subject, the apparatus comprising:
a plurality of imaging sensors, wherein at least one of the plurality of imaging sensors is a time-of-flight imaging sensor; and
a processing device coupled to the plurality of imaging sensors, the processing device being adapted to:
receive data streams from the plurality of imaging sensors; extract time parameter data from the data streams;
identify a physiological parameter from the extracted time parameter data; and provide an indication of the physiological parameter from the extracted time
parameter data, and
wherein the plurality of imaging sensors further comprises a thermal imaging device.

2. The apparatus of claim 1, further comprising an illumination component to provide one or more of narrow frequency illumination or structured illumination.

3. The apparatus of claim 1, wherein the plurality of imaging sensors further comprises an RGB imaging device. 4. The apparatus of claim 3, wherein the processing device is adapted to combine distance data of the time-of-flight imaging sensor with temperature data of the thermal imaging device and further with color data of the RGB imaging device.

5. The apparatus of claim 1, wherein the processing device is adapted to identify a region-of-interest (ROI) based on the data stream received from the thermal imaging device, in order to extract the time parameter data from the data stream received from at least one of the time-of-flight sensor and an RGB-sensor imaging device within the identified ROI. 6. The apparatus of claim 1, further comprising at least one microphone configured to receive audio data in an environment monitored by the plurality of imaging sensors.

7. The apparatus of claim 1, wherein, to extract time parameter data from the data streams, the processing device is further adapted to:
identify an edge in at least one of the data streams; and monitor motion characteristics of the detected edge.

8. The apparatus of claim 1, wherein the processing device is further adapted to:
determine signal to noise ratio of data in the received data streams; and
weight a first data stream higher than a second data stream based on the determined signal to noise ratios; and
combine extracted time parameter data from the data streams based on relative weights of the first data and second data to identify the physiological parameter.

9. The apparatus of claim 1, wherein, to extract time parameter data, the processing device is further adapted to perform pattern recognition to determine movement of an identified pattern in the received data streams.

10. The apparatus of claim 9, wherein the processing device is adapted to perform pattern recognition by using artificial intelligence.

11. The apparatus of claim 1, wherein the processing device is further adapted to filter a first data stream of one imaging sensor of the plurality of image sensors based on a frequency of respiration identified in a second data stream of a second imaging sensor of the plurality of image sensors.

12. The apparatus of claim 1, wherein the physiological parameter is one or more of respiration rate or heart rate.

13. The apparatus of claim 1, wherein the apparatus for monitoring physiological parameters of a subject is adapted to monitor the physiological parameters without contacting the subject.

14. A method for monitoring physiological parameters of a subject, the method comprising:
receiving, by a processing device, a plurality of data streams from each of a plurality of imaging sensors, wherein at least one of the imaging sensors is a time-of-flight imaging sensor;
extracting time parameter data from the data streams;
identifying, by the processing device, a physiological parameter from the extracted time parameter data; and
providing an indication of the physiological parameter from the extracted time parameter data,
wherein the plurality of imaging sensors further comprise a thermal imaging device.

15. A computer program product directly loadable into the internal memory of a digital computer, comprising software code portions for performing at least the following steps, when said product is run on a computer;
receiving, by a processing device, a plurality of data streams from each of a plurality of imaging sensors, wherein at least one of the imaging sensors is a time-of-flight imaging sensor;
extracting time parameter data from the data streams;
identifying, by the processing device, a physiological parameter from the extracted time parameter data; and
providing an indication of the physiological parameter from the extracted time parameter data,
wherein the plurality of imaging sensors further comprise a thermal imaging device.

16. The apparatus of claim 10, wherein the processing device is adapted to perform pattern recognition by using deep learning.

17. The method as claimed in claim 14, wherein an apparatus is used for the monitoring of the physiological parameters of the subject, the apparatus comprising:
the plurality of imaging sensors; and
the processing device coupled to the plurality of imaging sensors,
wherein the time parameter data is extracted from the data streams by the processing device; and
wherein the indication of the physiological parameter from the extracted time parameter data is provided by the processing device.
